SEQUENCE LISTING (1 sequence)

SEQ ID NO: 1 - DNA, 21 bp, Artificial Sequence
  Description of Artificial Sequence Synthetic oligonucleotide
ccccccgggggggtggggggg

(19) United States
(12) Patent Application Publication
(10) Pub. No.: US 2011/0159530 A1
(43) Pub. Date: Jun. 30, 2011

(54) METHODS OF QUANTIFYING BIOMARKERS

(75) Inventors: Kenneth A. Pass, Glenmont, NY (US); Martin Sorette, Amsterdam, NY (US); Barbara Shepard, Albany, NY (US)

(73) Assignee: Health Research, Inc.

(21) Appl. No.: 12/997,247

(22) PCT Filed: Jun. 10, 2009
(86) PCT No.: PCT/US09/46947

§ 371 Date: Dec. 9, 2010

Related U.S. Application Data
(60) Provisional application No. 61/060,647, filed on Jun. 11, 2008.

(51) Int. Cl. C12Q 1/28; G01N 33/72

(52) U.S. Cl. 435/28; 436/501

(57) ABSTRACT

Methods are provided for simultaneously measuring hematocrit (hct) level and the concentration of a biomarker in a blood specimen. Thus, serum biomarker concentrations can be more accurately measured. The methods are particularly useful for newborn screening programs.

### CROSS-REFERENCE TO RELATED APPLICATIONS

This application claims priority to U.S. Provisional Application No. 61/060,647 filed Jun. 11, 2008, the disclosure of which is incorporated here by reference in its entirety.

### BACKGROUND

In the first 15 years of newborn screening using a dried blood specimen (Guthrie spot), the assays provided a semi-quantitative result that was evaluated by a technician comparing the observed result with a series of controls with known values. With the introduction of RIA testing for thyroid disease in 1978, however, it became necessary to know the hematocrit (hct) in order to calculate the amount of biomarker thyroxin (T4) in the specimen. A CDC conference that year addressed the issue, noting that “the hct had little or no effect on serum volume in a ⅛-inch punch.” (Proceedings of a Conference on a National Model for Standardization of Neonatal Hypothyroid Screening Programs, Atlanta, Centers for Disease Control (1978).) Consequently, it was decided that a value of 55% would be used in all calculations derived from Guthrie spot analysis. Most reports, even continuing to the present, adjust the hct to 55% in materials prepared as standards and controls in the study. The CDC has adjusted its standards and controls to a 55% hct since 1978.

Twenty-three years later, the CDC newborn screening unit, now a robust QC/QA program for newborn screening throughout the world, published a report, showing that the amount of serum in the Guthrie spot was directly proportional to the hct of the infant (Mei, J V, et al., J. Nutr. 131:1631 S-1636S (2001)). The report further noted that as the volume of blood applied to the special testing paper increased, the amount of serum in the center of the ½ inch circle was increased.

Subsequently, many authors have addressed this issue, most recently in 2006, when Holub et al. showed that the hct of the blood applied to the special paper used for this purpose and the location of the ⅛ inch punch taken for analysis were significantly different from 55% and could produce, separately and in combination, incorrect analytical results (Holub, M., et al., Clin. Chim. Acta 373:27-31 (2006)). Earlier in 1995, an attempt was made to correct for the varying hcts by measuring sodium in a separate punch from the specimen (Arends, J., et al., Screening 4:101-105 (1995)). The study demonstrated that, when using the accepted cut-off for normal thyroxine (T4) of 10% with an adjustment applied for hct, seven of 17 positive cases would be detected that had been classified as normal without the adjustment. Analyzing a sample from a separate punch can introduce variability, however.

Thus, there is a need for a method for estimating, from a single punch, a biomarker's concentration and the hct in a Guthrie sample.

### SUMMARY

In one aspect, a method is provided for determining a concentration of a biomarker in the serum of a patient, the method comprising (a) obtaining a blood sample from a patient, (b) obtaining an extract from the blood sample, (c) measuring a concentration of hemoglobin (Hb) in the extract, (d) calculating a hematocrit (hct) value for the blood sample from the measured concentration of Hb, (e) measuring a concentration of a biomarker in the extract, and (f) determining a concentration of the biomarker in the serum of the patient using the calculated hct value, volume of the blood sample, and the measured concentration of biomarker in the extract.

In another aspect, a method is provided for calculating a concentration of a biomarker in the serum of a patient, comprising (a) measuring a concentration of Hb and a concentration of a biomarker in an extract obtained from a dried blood sample from a patient; (b) converting the measured concentration of Hb to a hct value; and (c) calculating a concentration of the biomarker in the serum of the patient using the hct value and the measured concentration of the biomarker in the extract.

In yet another aspect, a method is provided for arriving at concentrations of one or more biomarkers in a patient's whole blood, comprising (a) obtaining a punch from a dried whole blood specimen of a patient, wherein the dried whole blood specimen is made from a whole blood sample taken from the patient; (b) obtaining an eluent from the punch; (c) measuring concentrations of one or more biomarkers and of Hb in the eluent or a diluent thereof; (d) estimating an hct fraction of the whole blood sample from the measured concentration of Hb; and (e) adjusting the measured concentrations of the one or more biomarkers based on the estimated hct fraction to arrive at concentrations of the one or more biomarkers in the serum fraction of the patient's whole blood.

In another aspect, a method for determining a concentration of Hb in a sample, the method comprising: (a) contacting the sample with a fluorogenic substrate for peroxidase and hydrogen peroxide, such that when Hb is present a fluorescent product is produced; (b) determining the amount of the fluorescent product; and (c) calculating the concentration of the Hb in the sample based upon the amount of the fluorescent product determined in (b).

In some embodiments, the biomarker is a metabolic biomarker, and is suitable for use in a newborn screening program. The biomarker also may be selected from the group consisting of T4, TSH, and an amino acid. In other embodiments, the measurement of Hb and/or the biomarker is conducted using an affinity-based assay, which can be an immunoassay. In some aspects, the concentration of Hb and/or the biomarker is measured using a multiplexed immunoassay.

In another aspect, a universal assay buffer is provided which is useful in carrying out a substantially simultaneous analysis of two or more biomarkers. Thus, in some aspects, an assay buffer for the substantially simultaneous analysis of two or more biomarkers is also provided, the buffer comprising, in an aqueous mixture: (i) tris(hydroxymethyl)aminomethane hydrochloride (Tris-HCl); (ii) sodium chloride (NaCl); (iii) one or more emulsifiers; (iv) bovine serum albumin (BSA); (v) polyethylene glycol (PEG); (vi) one or more preservatives; (vii) bovine globulin; and (viii) a testosterone derivative. In preferred embodiments, the buffer is substantially free of 8-anilino-1-naphthalenesulfonic acid or a salt thereof.

In some embodiments, the aqueous buffer comprises, in an aqueous mixture: (i) about 50 mM Tris-HCl; (ii) about 150 mM NaCl; (iii) about 0.02% Tween 40; (iv) about 1% BSA; (v) about 0.5% polyethylene glycol; (vi) a preservative; (vii) about 0.05% bovine globulin; (viii) about 0.5 mg/L danazol; and (ix) about 1 mg/L of a protease inhibitor; provided that the assay buffer does not include about 0.5 mg or more per liter of 8-anilino-1-naphthalenesulfonic acid or a salt thereof.

In yet another aspect, a method is provided for arriving at concentrations of two or more biomarkers in a serum portion of a whole blood sample taken from a patient, the method comprising: (a) obtaining one or more punches from a dried whole blood specimen of a patient, in which the dried whole blood specimen is made from a whole blood sample taken from the patient; (b) obtaining an eluent from the one or more punches using a universal buffer as an elution solvent; (c) measuring a concentration of each of two or more biomarkers and of hemoglobin (Hb) in the eluent or a diluent thereof, provided that the measurement of the concentration of each of the two or more biomarkers is carried out substantially simultaneously using a multiplexed affinity assay; (d) estimating a hematocrit (hct) value for the whole blood sample from the concentration of Hb measured in step (c); (e) using the estimated hct value to adjust the concentration of each of the two or more biomarkers measured in step (c) to arrive at concentrations of two or more biomarkers in a serum portion of the whole blood sample taken from the patient.

In another aspect, a method is provided for screening a patient for two or more disorders by using a multiplexed affinity assay to determine the concentrations of two or more biomarkers in a serum portion of a whole blood sample taken from the patient, the method comprising: (a) obtaining a punch from a dried whole blood specimen of a patient, in which the dried whole blood specimen is made from a whole blood sample taken from the patient; (b) obtaining an eluent from the punch using a universal buffer as an elution solvent; (c) measuring a concentration of each of two or more biomarkers and of total hemoglobin (Hb) in the eluent or a diluent thereof, provided that the measurement of the concentration of each of the two or more biomarkers is carried out substantially simultaneously using a multiplexed affinity assay; (d) estimating a hematocrit (hct) value for the whole blood sample from the concentration of total Hb measured in step (c); (e) using the estimated hct value to adjust the concentration of each of the two or more biomarkers measured in step (c) to arrive at adjusted concentrations of the two or more biomarkers in a serum portion of the whole blood sample taken from the patient; and (f) using the adjusted concentrations of the two or more biomarkers to determine whether the patient suffers from two or more disorders.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 graphically illustrates the linear correlation of Hb concentration with hct fraction (in %) for arterial blood samples. FIG. 1 is from Kokholm, G., Scand. J. Clin. Lab. Invest. Suppl. 203:75-86 (1990).

FIG. 2 graphically shows the theoretical effect of the hct of a blood sample on the serum concentrations of thyroxine (T4) and TSH. “FP” denotes false positive, while “FN” denotes false negative.

FIG. 3 graphically plots mean fluorescent intensity (MFI) as a function of the Hb concentration (mg/mL) for a series of Hb standards. The MFI values were obtained using a Luminex system in which Hb antibodies were covalently coupled to a single set of beads and used to measure the concentration of Hb in a series of Hb standards.

FIG. 4 presents standard curves for thyroxin (T4) and thyrotropin (TSH) derived using separate Luminex bead sets for each biomarker, as described in Example 4 for the congenital hypothyroidism (CH) validation assay. In each standard curve, mean fluorescent intensity (MFI) is plotted as a function of the biomarker concentration for a series of standards for the biomarker.

FIG. 5 presents the results of the analysis for thyroxin (T4) in the CH validation assay described in Example 5. The Figure presents data obtained using the Luminex system for sample 1038, a residual newborn sample, in fifteen replicate plates, using the controls routinely provided by the Centers for Disease Control (CDC). The Luminex results are compared with results from the newborn screening program. For the Luminex analysis results, each well in the plates contained a mixture of the thyroxin and TSH assays developed separately and then combined for analysis of a single specimen. “C1” refers to the low control from the CDC for thyroxine, while “C2” refers to the high control.

FIG. 6 presents the results of the analysis for TSH in the CH validation assay described in Example 5. The Figure presents data obtained using the Luminex system for sample 1038, in 15 replicate plates, using the controls routinely provided by the CDC. The Luminex results are compared with results from the newborn screening program. Each well in the plates contained a mixture of the thyroxin and TSH assays developed separately and then combined for analysis of a single specimen. “C1” refers to the low control from the CDC for TSH, while “C2” refers to the high control.

FIG. 7 presents performance parameters for the thyroxin and TSH results obtained for the CH validation assay described in Example 5, using the Luminex assay. The performance parameters are presented using the three-level CDC controls.

FIG. 8 presents performance parameters for the thyroxin and TSH Luminex assay results from the CH validation assay described in Example 5 and compares them with results obtained by the newborn screening program.

FIG. 9 presents standard curves for the two biomarkers immunoreactive trypsin isoforms 1 and 2 (IRT1 and IRT2) derived using separate Luminex bead sets for each biomarker, as described in Example 5 for the cystic fibrosis (CF) validation assay. In each standard curve, mean fluorescent intensity (MFI) is plotted as a function of the biomarker concentration for a series of standards for the biomarker.

FIG. 10 shows the performance of the combined IRT1/2 bead sets from the Luminex assay described in Example 5 for the cystic fibrosis (CF) validation assay. The Table compares the Luminex assay results with results from the newborn screening program.

FIG. 11 presents a standard curve for the 17OHP biomarker, as described in the congenital adrenal hyperplasia (CAH) validation assay described in Example 5. The standard curve was derived using a Luminex bead set for 17OHP. In the standard curve, mean fluorescent intensity (MFI) is plotted as a function of the 17OHP concentration, for a series of 17OHP standards.

FIG. 12 graphically presents the results of a correlation study of the 17OHP assay results obtained using the Luminex bead set assay and the assay currently used in newborn screening. In the Figure, values obtained using the Luminex assay (y axis) are plotted as a function of the corresponding values obtained using the currently used assay (x axis).

FIG. 13 presents a table displaying the results of four specimens analyzed by the Example 5 multiplex biomarker assay to identify congenital hypothyroidism (CH), cystic fibrosis (CF), and congenital adrenal hyperplasia (CAH), as described in Example 5. The Luminex data is compared to similar results obtained in the three separate assays currently used to screen newborns.

FIG. 14 presents a standard curve for use in determining hematocrit using a single Luminex bead set detecting total hemoglobin. (left side), as described in Example 5. Results are also presented for three constructed hematocrit levels, which are compared with results obtained using Drabkins Reagent.

FIG. 15 graphically compares the IRT1+IRT2 (IRT1-IRT2) screen negative by the IRT1 and IRT2 assay with the IRT-R (reference IRT), as discussed in Example 6.

FIG. 16 graphically illustrates the population distribution of the 597 study samples discussed in Example 6.

### DETAILED DESCRIPTION

Methods have been developed for accurately determining the concentration of one or more biomarkers in the serum of a patient by taking into account the effect of the hematocrit (hct). The methods involve measuring the concentration of hemoglobin (Hb) in an extract of a specimen of a subject's blood, which also is used to measure the concentration of a biomarker. The Hb concentration is used to calculate a hct value for the specimen, which in turn is used to calculate the subject's true level of the biomarker. As the inventive methods more accurately measure serum biomarker concentrations, they represent an improvement over prior diagnostic assays that assign a value for the hct and will greatly benefit programs such as newborn screening.

In the methods, an extract is obtained from a sample of a patient's blood, and the concentration of Hb in the extract is measured. The measured Hb then is used to calculate a hct value for the blood sample. Meanwhile, the concentration of one or more biomarkers in the extract is measured. The hct value is then used to calculate the concentration of the biomarker in the patient's serum or plasma, or in the patient's whole blood.

The term “patient” or “subject” as used herein refers either to a human or to a non-human mammal. Examples of non-human mammals include, but are not limited to, primates, farm animals such as horses, sheep, or cattle, and domestic animals such as dogs, cats and the like.

In one aspect, the methods are used to measure biomarker concentrations in the blood of human patients, in particular, human infants less than about 6 months of age or, more typically, human infants one day to two weeks old. In one embodiment, the methods are used to measure concentrations of one or more biomarkers in the serum of human infants within one or two weeks of birth, e.g., to screen human newborns for inborn errors of metabolism. In another aspect, the methods can be used in veterinary applications to measure the concentrations of one or more biomarkers in the blood of non-human patients, including primates, or domestic animals such as farm animals and pets, including dogs and cats.

The terms “plasma” and “serum” as used herein refer to the liquid component of whole blood in which the blood cells are suspended. Both plasma and serum are clear, yellowish fluids that contain proteins, salts, sugars, vitamins, waste products. “Plasma” refers to the liquid component of blood before clotting has taken place, and which contains fibrinogen and other clotting elements. “Serum” denotes the liquid component of blood that remains after clotting, which lacks clotting elements and does not clot.

The terms “blood” and “whole blood” as used herein refer to plasma combined with blood cells (e.g., red blood cells, white blood cells, and platelets).

Both dried samples of blood and non-dried samples can be used in the methods. Examples of a non-dried blood sample include, but are not limited to, a liquid sample of blood such as a blood sample freshly obtained from a patient. Examples of dried blood samples include, but are not limited to, Guthrie spots (dried blood spots on filter paper).

In one embodiment, dried blood samples are used. In one example, the dried blood sample is in the form of a Guthrie spot. Methods for preparing Guthrie spots are known to those of skill in the art, e.g., clinicians and those with training in the field of medicine. Typically, the dried blood on the Guthrie spot is analyzed by first obtaining a ⅛-inch diameter disk (−3.2 mm in diameter) from the Guthrie spot (i.e., the disk is punched from the Guthrie spot), and then extracting the blood on the disk with an elution buffer to obtain an extract. The volume of blood on each ⅛-inch disk is generally estimated to be about 3 microliters.

In some embodiments, the methods employ Guthrie spots to measure the concentrations of a series of biomarkers in the serum or plasma of human infants to detect inborn errors of metabolism and the like.

In some aspects, a liquid blood sample is used for testing, e.g., for “point of care” screening of newborns. In these embodiments, the amount of blood for use in each sample can readily be determined by one of skill in the art. Typically, liquid blood sample volumes are larger, typically three to five milliliters, than those of dried blood samples. For example, in some embodiments, the volume of the blood sample that is tested is equivalent to about 3 microliters, the volume of blood generally estimated to be present on a ⅛ inch diameter disk punched from a dried blood Guthrie spot.

Once a patient sample is secured, an extract of the patient's blood is obtained. In some embodiments, the extract is taken from a dried blood sample, e.g., a Guthrie spot, using an elution buffer. Extracts of dried blood from Guthrie spots are typically obtained by punching a ⅛ inch diameter disk from the Guthrie spot and incubating the disk with about 100 uL of elution buffer for about 30 minutes, sometimes with sonication. Extracts of liquid blood samples are obtained similarly, by incubating an appropriate volume of liquid blood, generally about 3-5 milliliters, in an appropriate volume of elution buffer, optionally with agitation. After elution, the resulting extract typically is filtered to remove solid particles.

Examples of suitable elution buffers include, but are not limited to, PBS (phosphate buffered saline) optionally combined with one or more emulsifiers (e.g., Tween-20 or another detergent) at various pH values, including those at or close to physiological pH (e.g., at pH 7.4).

For example, elution of dried blood or liquid blood samples can be performed using a universal assay buffer, which, in some embodiments, comprises in an aqueous mixture: (i) tris(hydroxymethyl)aminomethane hydrochloride (Tris-HCl); (ii) sodium chloride (NaCl); (iii) one or more emulsifiers; (iv) bovine serum albumin (BSA); (v) polyethylene glycol (PEG); (vi) one or more preservatives; (vii) bovine globulin; and (viii) a testosterone derivative. Preferably, the universal assay buffer is substantially free of 8-anilino-1-naphthalenesulfonic acid or a salt thereof. Preferably, the Tris-HCl is present at a concentration falling in the range of about 25 to about 75 mM, more preferably, about 50 mM and that the pH of the buffer is adjusted to a pH of less than about 8, preferably falling in the range of about 7 to about 8, more preferably about 7.8 and most preferably about 7.75. Moreover, the NaCl is preferably present at a concentration falling in the range of about 100 mM to about 200 mM, more preferably, about 150 mM.

In a particular embodiment, the one or more emulsifiers in the universal assay buffer may comprise any one or more detergents. Suitable emulsifiers/detergents include polyoxyethylene sorbitan monopalmitate (a/k/a Tween 40), polyoxyethylene sorbitan monooleate (a/k/a Tween 80), and the like or combinations thereof. Preferably, the Tween 40 is used as the emulsifier and the chosen emulsifier is present at a concentration falling in the range of about 0.001% to about 0.1%, more preferably, about 0.02%. Preferably, the BSA is present at a concentration of about 0.5% to about 2%, more preferably, about 1%.

The preferred universal assay buffer also comprises PEG, preferably PEG 6000. The PEG may be present at a concentration falling in the range of about 0.1% to about 1%, preferably, about 0.5%. Likewise, a bovine globulin is included, which may be present at a concentration falling in the range of about 0.01% to about 0.1%, preferably, about 0.05%. As described above, the inventive universal assay buffer includes a testosterone derivative. Examples of suitable testosterone derivatives include, but are not limited to, 17α-ethynyltestosterone (ethisterone), 17β-hydroxy-2,4,17α-pregnadien-20-yno[2,3-D] isoxazole (danazol), 19-nor-17α-ethynyltestosterone (norethindrone), or combinations thereof. A preferred testosterone derivative comprises danazol, and the chosen testosterone derivative may be present at a concentration ranging from about 0.1 mg per liter to about 1 mg per liter, preferably, about 0.5 mg per liter.

In one embodiment, the universal assay buffer contains one or more preservatives, which may preferably be selected from (but not limited to) sodium azide present at a concentration ranging from about 0.01% to about 0.1%, preferably, about 0.05%, albumin present at a concentration ranging from about 0.1% to about 2%, preferably, about 1%, or a combination thereof. What is more, a preferred universal assay buffer includes a protease inhibitor, such as (but not limited to) aprotinin, which may be present at a concentration ranging from about 0.1 mg per liter to about 2 mg per liter, more preferably, about 1 mg per liter, tranexamic acid or a salt thereof, which may be present at a concentration ranging from about 0.1 mg per liter to about 2 mg per liter, more preferably, about 1 mg per liter, or a combination thereof.

Accordingly, in some embodiments, the elution buffer is a universal assay buffer comprising (in a water mixture): (i) about 50 mM Tris-HCl; (ii) about 150 mM NaCl; (iii) about 0.02% Tween 40; (iv) about 1% BSA; (v) about 0.5% polyethylene glycol; (vi) a preservative; (vii) about 0.05% bovine globulin; (viii) about 0.5 mg/L danazol; and (ix) about 1 mg/L of a protease inhibitor; provided that the buffer does not include about 0.5 mg or more per liter of 8-anilino-1-naphthalenesulfonic acid or a salt thereof, if at all. In one embodiment, the preservative comprises about 0.05% sodium azide. In another embodiment, the protease inhibitor comprises about 1 mg/L aprotinin. An in still another embodiment, the buffer has a pH falling in the range of about 7 to about 8.

Once an extract is obtained from the blood sample, the extract is then analyzed to determine the concentration of Hb and biomarker(s). Any biomarker can be measured. Examples include, but are not limited to, amino acids, acylcarnitines, hormones such as thyroxine and thyrotropin, See Holub et al., Clin. Chim. Acta 373:27-31 (2006), and Mei et al., J. Nutr. 131:1631 S-1636S (2001), which are hereby incorporated by reference. The concentrations of many of these biomarkers are measured in newborn screening programs to determine the presence of inborn errors of metabolism. See, e.g., the list of conditions tested in the newborn screening program conducted by the New York State Department of Health (www.wadsworth.org/newborn/babhealth.htm).

Suitable methods for measuring the amount of Hb and biomarker(s) in the blood sample extract are known to those of skill in the art and include, but are not limited to, chromatographic methods, such as HPLC (high performance liquid chromatography), tandem mass spectrometry, and affinity-based methods, such as immunoassays. Methods for calculating the concentration of an analyte (e.g., Hb and biomarkers) from measurements of the amount of the analyte from each of these methods are also well known in the art.

In some embodiments, the extract is analyzed using an immunoassay. Immunoassays are well known in the art and can be performed using different formats, including a competitive or non-competitive (“sandwich”) format, and other variations known in the art. In some embodiments, the immunoassay can be performed using either a competitive or non-competitive (“sandwich”) format, or both formats. For example, when multiple biomarkers are measured, either a competitive format or a sandwich format can be used, so that different biomarkers can be measured using different formats in the same assay. In some embodiments, one biomarker can be measured using a competitive immunoassay format, while a second biomarker in the same assay can be measured using a sandwich-type immunoassay format. In other embodiments, the same assay format is used for each biomarker analyzed. In one aspect, an array can be used. One example is a reverse-phase protein microarray.

The detection antibody used in an immunoassay is labeled with a detectable label, such as an enzymatic or fluorescent label, or a radioisotope. Methods of detecting the labeled antibody are well known in the art. Examples, include but are not limited to, colorimetric, chemiluminescent, radiometric, or fluorometric methods.

In some embodiments, the detection antibody is labeled with a fluorescent probe, which is detected using fluorometric methods known in the art. A wide range of fluorescent probes are commercially available (See, e.g., Invitrogen Corporation, Carlsbad, Calif.). Examples of suitable fluorescent probes include, but are not limited to, phycoerythrin, including phycoerythrin-streptavidin and phycoerythrin-avidin conjugates. In addition, methods and reagents for coupling fluorescent probes to proteins, including antibodies, are well known in the art. See, for example, technical handbooks from Invitrogen Corporation (Carlsbad, Calif.) and Pierce (Thermo Fisher Scientific, Inc., Rockford, Ill.).

A variety of antibodies to biomarkers are also commercially available. See, for example, Sigma-Aldrich (St. Louis, Mo.), Invitrogen Corporation (Carlsbad, Calif.), and BD Biosciences (San Jose, Calif.). Antibodies suitable for use in the disclosed methods can be chosen readily by those skilled in the art.

In some embodiments, the extract of the patient's blood sample is tested for multiple biomarkers using a multiplex assay format. In one example, multiplexed immunoassays are used. Examples of multiplexed immunoassays include, but are not limited to, immunoassay-based protein microarrays, tandem mass spectrometry, and flow cytometric techniques.

For example, the amounts of Hb and biomarkers in the patient's blood sample can be measured using an assay system from Luminex Corporation (Austin, Tex.), such as XMAP. The assay system uses five-micron polystyrene beads that have been impregnated with a precise ratio of two fluorescent dyes, creating 100 spectrally identifiable beads. The surface of these beads is coated with carboxyl terminals (an estimated one million) which serve as the attachment point for the immunoassay that is built on the beads. Using the principles of traditional immunoassay, a sandwich or competition assay is developed for the target biomarker. At the completion of the four-hour assay, the beads are run through a modified flow cytometer. Two lasers query the beads: one for its ID number; the second for the intensity of the phycoerythrin signal resulting from the immunoassay. Up to 100 beads for each biomarker are counted, then averaged to record the MFI (mean fluorescent intensity) for that assay. Since multiple beads sets can be used simultaneously in the assay, the benefits of multiplexing can be utilized, allowing multiple biomarkers for a condition (e.g., thyroxine and thyrotropin for congenital hypothyroidism) and/or multiple conditions to be analyzed substantially simultaneously. See Bellisario, R., et al., Early Hum. Dev. 64:21-25 (2001), and Bellisario, R., et al., Clin. Chem. 46(9):1422-1424 (2000).

In some aspects, evaluating a sample for multiple biomarkers can improve the accuracy of detecting whether a patient has, or may develop, a particular disorder. For example, when evaluating a patient sample for congenital hypothyroidism, a combination of biomarkers TSH and T4 can be used. Similarly, when testing for congenital adrenal hyperplasia, a combination of markers 17OHP and cortisol can be used. When measuring for cystic fibrosis, a combination of markers IRT1 and IRT2 can be used.

In other aspects, a sample is evaluated for the presence of multiple biomarkers to ascertain whether a patient has, or may develop, one or more of a variety of disorders. For instance, a sample can be evaluated for biomarkers indicative of disorders such as sickle cell disease, sickle cell trait, human immunodeficiency virus, homocystinuria, hypermethioninemia, branched-chain ketonuria, phenylketonuria, tyrosinemia, carnitine-acylcarnitine translocase deficiency, carnitine palmitoyltransferase I deficiency, carnitine palmitoyltransferase II deficiency, carnitine uptake defect, 2,4-dienoyl-CoA reductase deficiency, long-chain hydroxyacyl-CoA dehydrogenase deficiency, medium-chain acyl-CoA dehydrogenase deficiency, medium-chain ketoacyl-CoA thiolase deficiency, medium/short-chain hydroxyacyl-CoA dehydrogenase deficiency, mitochondrial trifunctional protein deficiency, multiple acyl-CoA dehyrdogenase deficiency, short-chain acyl-CoA dehyrdogenase deficiency, very long-chain acyl-CoA dehydrogenase deficiency, cobalamin A,B cofactor deficiency, cobalamin C,D cofactor deficiency, glutaric acidemia type I, 3-hydroxy-3-methylglutaryl-CoA lyase deficiency, isobutyryl-CoA dehydrogenase deficiency, isovaleric acidemia, malonic acidemia, 2-methylbutyryl-CoA dehydrogenase deficiency, 3-methylcrotonyl-CoA carboxylase deficiency, 3-methylglutaconic acidemia, 2-methyl-3-hydroxybutyryl-CoA dehydrogenase deficiency, methylmalonyl-CoA mutase deficiency, mitochondrial acetoacetyl-CoA thiolase deficiency, multiple carboxylase deficiency, propionic acidemia, argininemia, argininosuccinic acidemia, citrullinemia, hyperammonemia/hyperornithinemia/homocitrullinemia, biotinidase deficiency, galactosemia or Krabbe disease.

It is important to note that in some cases, it is desirable that at least one of the two or more biomarkers comprises a protein biomarker, which is not a cytokine What is more, the two or more non-cytokine protein biomarkers may include, but are not limited to, thyroxine (T4), thyrotropin (TSH), 17-hydroxyprogesterone (17OHP), an isoform of immunoreactive trypsin (IRT1), an isoform of immunoreactive trypsin (IRT2), cell surface receptor (CD3), a cell surface receptor (CD45), thyroxine binding globulin (TBG), pancreatic associated protein (PAP), as well as combinations thereof. In select cases, the universal assay buffer is useful for the substantially simultaneous analysis of two or more biomarkers, including 17OHP and a steroid or a steroid derivative, including cortisol, androsteindione, and the like. In still other cases the universal assay buffer is useful for the substantially simultaneous analysis of two or more biomarkers, including CD3, CD45 and a cytokine (IL-7).

Thus, in some embodiments, the present method also encompasses a method of arriving at concentrations of two or more biomarkers in a serum portion of a whole blood sample taken from a patient, the method comprising: (a) obtaining one or more punches (preferably a single punch) from a dried whole blood specimen of a patient, in which the dried whole blood specimen is made from a whole blood sample taken from the patient; (b) obtaining an eluent from the one or more punches using an universal assay buffer as an elution solvent; (c) measuring a concentration of each of two or more biomarkers and of hemoglobin (Hb) in the eluent or a diluent thereof, provided that the measurement of the concentration of each of the two or more biomarkers is carried out substantially simultaneously using a multiplexed affinity assay; (d) estimating a hematocrit (hct) value for the whole blood sample from the concentration of Hb measured in step (c); (e) using the estimated hct value to adjust the concentration of each of the two or more biomarkers measured in step (c) to arrive at concentrations of two or more biomarkers in a serum portion of the whole blood sample taken from the patient.

In particularly preferred embodiments of the method, the eluent in (b) is obtained from a single punch of the dried whole blood specimen in (a). In these embodiments, multiple biomarkers are assayed using an eluent obtained from a single punch (e.g., a single 3 mm punch) taken from a single dried blood specimen (e.g., Guthrie spot). Using eluent from a single punch reduces variability in the measurement of the biomarker concentrations, which can occur when two or more punches are used from the same dried blood specimen (same Guthrie spot). Such variations are often be due to slight differences in biomarker concentration that occur when the blood sample is spotted onto the center of the filter paper used to make the Guthrie spot and subsequently “spreads out” on the paper.

A suitable multiplexed affinity assay may be one comprising a multiplexed bead-based affinity assay, a multiplexed electroluminescence affinity assay, a multiplexed chemiluminescence affinity assay, and the like, or combinations thereof. If desired the affinity assay may include an immunoassay. Preferably, at least one of the two or more biomarkers comprises a protein biomarker. And more preferably still, at least one of the two or more biomarkers comprises a protein biomarker, which is not a cytokine.

In certain cases, a preferred method further comprises determining if the estimated hct value is aberrant. Such aberrant hct values may arise, for example, because the affected patient either suffered or is suffering from anemia. These possibilities should preferably be considered.

In some embodiments, a universal assay buffer is contemplated for use in the multiplexed assay. In these embodiments, the universal assay buffer may comprise, in an aqueous mixture: (i) tris(hydroxymethyl)aminomethane hydrochloride (Tris-HCl); (ii) sodium chloride (NaCl); (iii) one or more emulsifiers or detergents; (iv) bovine serum albumin (BSA); (v) polyethylene glycol (PEG); (vi) one or more preservatives; (vii) bovine globulin; (viii) danazol; and (ix) a protease inhibitor; provided that the assay buffer does not include about 0.5 mg or higher per liter of 8-anilino-1-naphthalenesulfonic acid (ANSA) or a salt thereof, if any. In certain cases, however, it may be desirable to also include (or replace the danazol altogether) with as little as about 100 μg per liter or so of ANSA. In another embodiment, the universal assay buffer (which again is also the eluent used to elute the two or more biomarkers and Hb from the one or more punches, preferably, a single punch) has a pH of about 7.8 and consists essentially of: (i) about 50 mM Tris-HCl; (ii) about 150 mM NaCl; (iii) about 0.02% Tween 40; (iv) about 1% BSA; (v) about 0.5% polyethylene glycol-6000; (vi) about 0.05% sodium azide; (vii) about 0.05% bovine globulin; (viii) about 0.5 mg/L danazol; and (ix) about 1 mg/L aprotinin. Preferably, at least one of the two or more biomarkers is a non-cytokine biomarker. More preferably, the non-cytokine biomarker includes, but is not limited to, T4, TSH, 17OHP, cortisol, androsteindione, IRT1, IRT2, CD3, CD45, TBG, PAP, or combinations thereof.

It may also be advantageous to carry out the step of using the estimated hct value to adjust the concentration of each of the two or more biomarkers measured in step (c) above with the aid of a processor and/or a computer algorithm configured for such purpose. While the method described has many applications, one particularly desirable application relates to the carrying out of the method as part of a newborn screening program. Hence, a suitable patient may include, but is not limited to, a human infant.

The concentration of Hb in the extract of the patient's blood sample is also determined and then used to estimate a hct value for the original blood sample. As discussed above, using the hemoglobin (Hb) concentration to estimate a hct value for the blood sample allows for the more accurate measurement of serum biomarker concentrations in the disclosed methods.

Measurement of the Hb in the extract also provides additional advantages to the disclosed methods. For example, measuring the extract of the patient's blood sample for Hb allows for the identification of individual Hb variants. As a result, in addition to screening a blood sample for various biomarkers, the present methods can be used to screen the sample for the presence of one or more specific Hb variants that can be used to identify certain blood disorders, such as sickle cell anemia, that are associated with the variants. Thus, in some embodiments, the present method can be used to determine whether a subject or patient has, or may develop, a blood disorder. In some embodiments, the present method can also be used to determine whether a particular Hb variant is present in the patient's or subject's blood sample, or can be used to measure the concentration of, or the presence of, a particular Hb variant in the patient's or subject's blood sample.

Measurement of the concentration of Hb in the extract can serve also as a positive control for the assay by providing assurance that the extract is physically present in the assay apparatus (e.g., ruling out operator or machine error) and that the assay chemistry has performed as expected (e.g., that all assay components were delivered to the sample). For example, measuring a low or non-existent Hb concentration for a sample would be an indication that the assay is not performing properly for the sample, or that the sample was absent. Thus, in some embodiments, the present method can be used to provide an improved method (e.g., a more reproducible, reliable, and/or more accurate method) of determining the concentration(s) of one or more biomarkers in a blood sample of a patient or subject, by measuring both the concentration of Hb, or one or more Hb variants, and the concentration(s) of the one or more biomarkers, in an eluent or diluent thereof of a sample of a patient's blood.

The term “hematocrit” as used herein refers to the proportion of blood volume that is occupied by red blood cells. The hct may be estimated by centrifuging heparinized blood in a capillary tube to separate the blood into layers, and dividing the volume of packed red blood cells by the total volume of the blood sample.

Hemoglobin (Hb) is measured as described above. In some embodiments of the method, Hb is measured using the same assay format and method of detection that is used for one or more of the biomarkers. For example, Hb and one or more biomarkers can be measured using flow-cytometry, such as in an assay system from Luminex Corporation (Austin, Tex.), in which, for example, Hb and each of the biomarkers are detected using antibodies specific for Hb and each biomarker. In some of these embodiments, both Hb and biomarkers are measured using a fluorescent probe bound to the detection antibody. Using the amount of Hb measured in the extract, a Hb concentration is then calculated for the original blood sample.

In some of these embodiments, for example, the Hb concentration can be measured using a pan-hemoglobin antibody. In other embodiments, the Hb concentration can be measured by measuring the concentrations of individual Hb variants to arrive at a total Hb concentration. For example, in some embodiments, individual antibodies to each of a series of Hb variants can be used to measure the concentration of total Hb, rather than one pan-hemoglobin antibody which detects all Hb variants. In some of these embodiments, the concentrations of four or more individual variants are measured to arrive at the concentration of total Hb. Typically, at least five individual Hb variants are measured. Examples of Hb variants include, but are not limited to, Hemoglobin A (Hb A), Hemoglobin F (Hb F), Hemoglobin S (Hb S), Hemoglobin E (Hb E), Hemoglobin C (Hb C), and Hemoglobin H (Hb H).

Measurement of one or more individual Hb variants not only allows for the estimation of a hct value for the original blood sample, but also makes possible the identification of the type of Hb present in the blood sample, which can be used to determine whether a patient has, or may develop, one or more blood disorders that are associated with specific Hb variants, such as, for example, sickle cell anemia, thalassemia, and hemolytic anemia.

Thus, is some embodiments, the extract of the patient's blood sample is tested for multiple biomarkers using a multiplex assay format, in which the concentrations of two or more biomarkers are measured (e.g., concentrations of the biomarkers T4, TSH, IRT1, IRT2, and 17OHP), in addition to measurement of the concentrations of four or more individual Hb variants (e.g., Hb A, Hb F, Hb S, Hb E, and Hb C). Measurement of these biomarkers allows one to determine at the same time whether the patient has, or may develop, one or more disorders associated with the biomarkers as well as those disorders associated with one or more of the individual Hb variants, such as sickle cell anemia, which is associated with Hb S.

In other embodiments, Hb is measured using an assay format, or a method of detection, that is different from that used for one or more of the biomarkers. For example, Hb can be measured using a separate assay. For example, Hb concentrations can be measured using an assay based on the pseudoperoxidase activity of heme. In this assay, the heme pseudoperoxidase activity catalyzes the conversion of a substrate to a fluorescent product in the presence of hydrogen peroxide. Acceptable substrates include, but are not limited to AMPLEX RED or AMPLEX ULTRARED (Invitrogen Corporation, Carlsbad, Calif.). In the presence of a peroxidase, the substrate reacts with hydrogen peroxide in a 1:1 stoichiometry to produce resorufin, a highly fluorescent product. See MOLECULAR PROBES handbook, Section 10.5 (Invitrogen Corporation, Carlsbad, Calif.). The fluorescent product in the assay is then measured using known fluorescence detection methods. For example, the resulting fluorescent product can be detected using fluorescent flow cytometry, such as the Luminex assay system described above. When the Luminex assay system is utilized, the fluorescent product (resorufin) can be detected using aptamer-conjugated microspheres that capture the fluorescent reaction product. Examples of suitable aptamers are disclosed in Asai et al., Nucl. Acids Res. (supplement 3):321-322 (2003).

Thus, in some embodiments, both biomarkers and Hb are measured using a fluorescence-based detection system, such as the Luminex system, in which one or more biomarkers are measured using a fluorescent probe bound to each biomarker-specific antibody, while Hb is measured using the pseudoperoxidase assay, as described above, in which aptamer-conjugated microspheres capture the fluorescent product (e.g., resorufin) produced by the pseudoperoxidase reaction of the heme in the Hb.

After the Hb concentration is calculated, the calculation is converted to an estimated hct value for the original blood sample using the following equation, which is taken from Kokholm, G., Scand. J. Clin. Lab. Invest. Suppl. 203:75-86 (1990) (the contents of which is herein incorporated by reference in its entirety):

xHct=0.0485+ctHb+0.0083

where “ctHb” represents Hb concentration (mm/L), and “xHct” represents the corresponding hct value. The hct value is then used to calculate the volume of serum in the original sample, which is used in the calculation of the concentration of the biomarker that is also measured from the same extract of the same blood sample. This allows for a more accurate determination of serum biomarker concentrations. Plasma biomarker concentrations can be calculated in a similar fashion. Concentrations of the biomarkers are calculated using methods known in the art that are appropriate for the individual assay format and detection method used.

While the calculations of the Hb, hct and biomarker concentrations can be estimated by hand, standard software typically provided with luminometers and the like can greatly facilitate the process.

### EXAMPLES

### Example 1

Antibodies against the Hb molecule were used to create an assay on a single set of beads to measure the Hb concentration in a Guthrie spot. An assay system from Luminex Corporation (Austin, Tex.) was used.

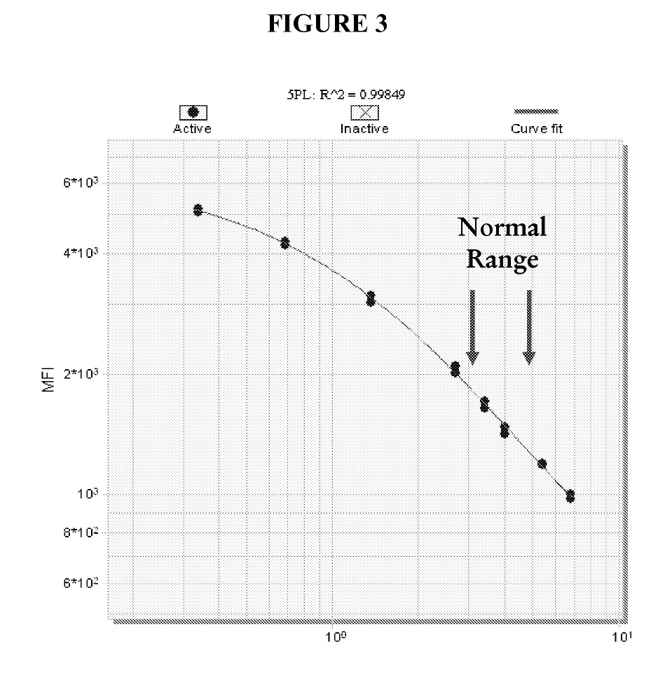

The standards shown in FIG. 3 were diluted 1:100 in assay buffer (phosphate buffered saline/0.2% gelatin). A 75 ul aliquot was applied to each well of a filter assay plate (Millipore MABVN1250, Millipore Corporation, Billerica, Mass.). 50 ul of human Hb labeled with biotin was applied to each well. Luminex microspheres conjugated with a pan-Hb antibody (25 ul) were applied and incubated for 60 minutes with shaking at 37° C. The beads were washed three times in phosphate buffered saline/0.05% Tween 20. Streptavidin PE (phycoerythrin conjugated to streptavidin) (100 ul, 4 ug/mL) was applied and incubated for 30 minutes with shaking Beads were washed one time in phosphate buffered saline/tween 20, resuspended in Luminex sheath fluid and analyzed on the Luminex system.

Mean fluorescent intensity (MFI) was measured for each Hb standard and plotted as a function of the Hb concentration (mg/mL) in the standard, as shown in FIG. 3. Curve fitting software (LIQUICHIP, Qiagen Inc., Valencia, Calif.) was used to fit the displacement curve.

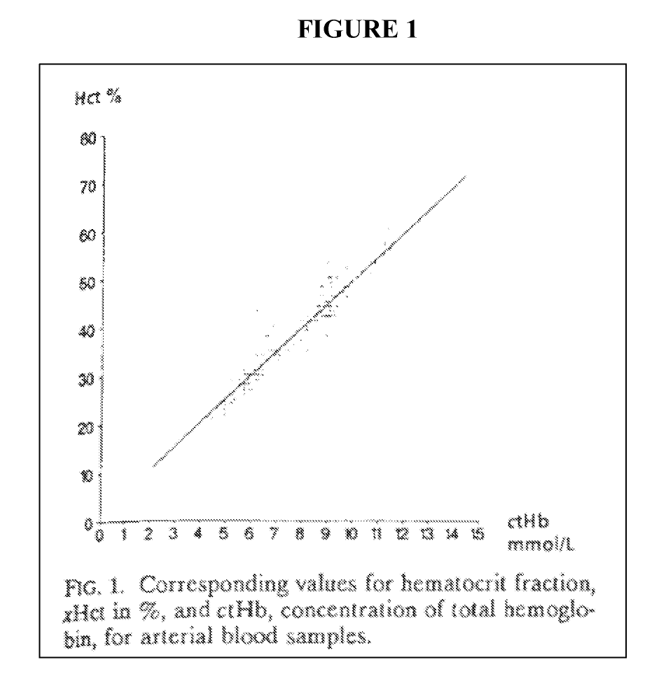

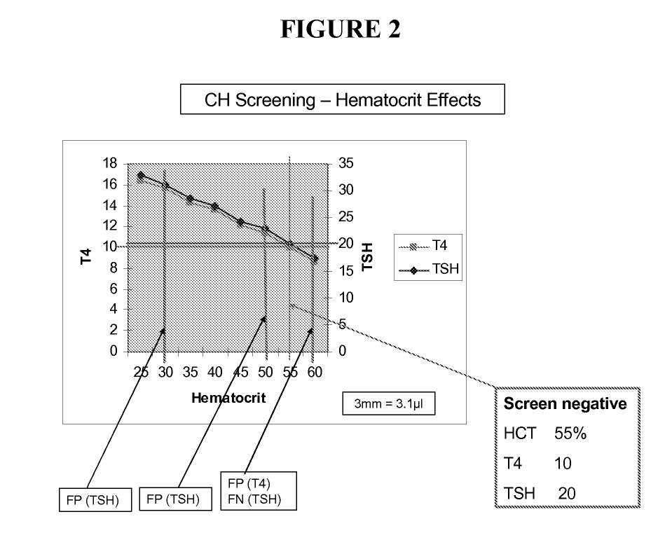

As shown in FIG. 3, the assay is linear across the range of normal Hb concentrations. A corresponding hct value can be obtained by converting the Hb concentration read from the plot (shown in FIG. 1) to its corresponding hct value using the following equation: xHct=0.0485+ctHb+0.0083, where “ctHb” represents Hb concentration (mm/L), and “xHct” represents the corresponding hct value. See Kokholm, G., Scand. J. Clin. Lab. Invest. Suppl. 203:75-86 (1990), which is herein incorporated by reference.

### Example 2

The pseudoperoxidase activity of Hb can be used as the basis of an alternative assay for measuring Hb. In one aspect, the assay utilizes the Luminex system (Luminex Corporation, Austin, Tex.), and uses a single set of beads to measure the Hb concentration in a sample.

A series of Hb standards are prepared in sample buffer. Separately, a set of Luminex aptamer-conjugated microsphere beads is prepared by covalently coupling an aptamer, for example 5′-CCCCCCGGGGGGGTGGGGGGG-3′, to the beads according to the manufacturer's instructions (Luminex Corporation, Austin, Tex.), or according to coupling procedures known in the art.

The Hb standards are prepared in assay buffer (phosphate buffered saline/0.2% gelatin), and a 75 ul aliquot of each diluted standard is applied to each well of a filter assay plate (Millipore MABVN1250, Millipore Corporation, Billerica, Mass.). Appropriate amounts of a fluorogenic substrate, such as AMPLEX RED (Invitrogen Corporation, Carlsbad, Calif.), and hydrogen peroxide are added to each well and incubated. During incubation, the reaction mixture in each well is protected from light.

The aptamer-conjugated beads (25 ul) are then applied to the wells containing the Hb standards and hydrogen peroxide, and incubated for 60 minutes with shaking at 37° C. The beads are then washed three times in phosphate buffered saline/0.05% Tween 20, and resuspended in Luminex sheath fluid for analysis in the Luminex system.

Mean fluorescent intensity (MFI) is measured for each Hb standard and plotted as a function of the Hb concentration (mg/mL) in the standard. Curve fitting software (LIQUICHIP, Qiagen Inc., Valencia, Calif.) is used to fit the displacement curve.

### Example 3

A sample of dried blood from a Guthrie spot is assayed for thyroxine (T₄) using two sets of fluorescent microsphere beads, one set to measure the concentration of T₄ in the dried blood sample and a second set to measure the Hb concentration.

The assay consists of two microtiter wells as follows: The first well contains one set of microspheres to detect the quantity of T₄ present in the Guthrie spot eluent according to the protocol outlined in Bellisario et al. (“Simultaneous Measurement of Thyroxine and Thyrotropin from Newborn Dried Blood-Spot Specimens Using a Multiplexed Fluorescent Microsphere Immunoassay,” Clin. Chem. 46(9):1422-1424 (2000), which is hereby incorporated by reference). The second well contains a second set of microspheres to quantify Hb using the same Guthrie spot eluent appropriately diluted (e.g., 1 part eluent:99 parts eluent buffer), as described in Example 1 above. A competitive-inhibition assay is used for measurement of T4, and a sandwich-capture assay format is used for measuring Hb.

Briefly, T₄-BSA antigen and anti-Hb (anti-Hb) monoclonal or polyclonal capture antibody are covalently coupled to two microsphere sets according to the manufacturer's instructions (Luminex Corporation, Austin, Tex.). Preferably, the polyclonal capture antibody is one with a pan-Hb specificity recognizing Hbs A1, A2, F and S and other Hb variants. Alternatively, multiple monoclonals to the various Hb variants can be utilized. The anti-Hb monoclonal or polyclonal antibody (e.g., 100 ug), and the T4-BSA antigen (e.g., 25 ug) are separately covalently attached to the carboxylate groups of two distinct microsphere sets using a two-step coupling method. In the first step, the microspheres (10⁷ microspheres) are activated with 0.25 mg of 1-ethyl-3-(3-dimethylaminopropyl)-carbodiimide hydrochloride and 0.25 mg of N-hydroxysulfosuccinimide (Sulfo-NHS) in 0.5 mL of 0.1 mol/L sodium phosphate buffer, pH 6.1, for 20 minutes at room temperature. The microspheres are centrifuged and resuspended in 0.5 mL of phosphate-buffered saline (PBS), pH 7.4. After a second wash, each protein is covalently coupled to its microsphere set in 0.5 mL of PBS, pH 7.4, by incubation for 2 hours at room temperature. The coupled microspheres are stored in PBS (pH 7.4) containing 10 g/L bovine serum albumin and 0.5 g/L sodium azide (storage buffer).

The anti-Hb monoclonal or polyclonal detection antibody and the anti-T4 monoclonal detection antibody are biotinylated separately according to the manufacturer's instructions. For example, each detection antibody is biotinylated with 40 ug of Sulfo-NHS-LC-Biotin (from Pierce) in 100 uL of PBS, pH 7.4, for 30 minutes at room temperature. The biotinylated antibodies are stored in PBS (pH 7.4) containing 10 g/L bovine serum albumin and 0.5 g/L sodium azide (storage buffer).

Calibration curves are first constructed for T₄ and Hb using T₄ and Hb dried blood-spot calibrators and controls, which are prepared as described in Reilly et al., Clin. Chem. 44(2):317-326 (1998), which is hereby incorporated by reference, and in Example 1 above. An approximately 3.2 mm (⅛ inch) diameter disk is punched from each dried blood-spot T₄ and Hb calibrator or control and eluted with 100 uL of phosphate buffered saline, pH 7.4, containing 0.05% Tween-20 and 0.2% gelatin, by sonication at room temperature for 30 minutes. The volume of blood per 3.2 mm disk is assumed to be about 3 microliters. The eluent is filtered in a 0.45 um centrifugal filter unit (Millipore Corporation, Billerica, Mass.).

For the assay, about 50 uL (−5000 microspheres) of the bound T₄-BSA-microsphere set and of the anti-Hb capture antibody-microsphere set are added to individual wells in a 96-well filter-bottom microtiter plate (Millipore Corporation, Billerica, Mass.), with each well containing only one set of microspheres. The microspheres in each well are washed with 200 uL of PBS (pH 7.4) containing 0.5 mL/L Tween 20 (PBS-Tween).

For those wells containing T₄-BSA-bound microspheres, filtered blood-spot eluent (e.g., 50 uL) and 50 uL of biotinylated anti-T₄ detection antibody (0.25 mg/L in storage buffer) are added to the T₄-BSA-bound microspheres and incubated at 37° C. for 30 minutes, with shaking. For those wells containing anti-Hb antibody-bound microspheres, the filtered blood-spot eluent is first diluted appropriately. The diluted eluent (50 uL) and 50 uL of biotinylated anti-Hb detection antibody (50 uL; 4 mg/L in storage buffer) then are added and incubated with the anti-Hb antibody-bound microspheres at 37° C. for 30 minutes, with shaking. After incubation, the microtiter plate is washed three times with 200 uL of PBS-Tween. Streptavidin R-phycoerythrin (Invitrogen Corporation (Carlsbad, Calif.)) (50 uL) is then added to each well and incubated with shaking at 37° C. for 15 minutes. A final wash is performed, and the microspheres are resuspended in 100 uL of PBS-Tween for analysis and data collection using a Luminex¹⁰⁰ instrument (Luminex Corporation, Austin, Tex.) in multiplexed acquisition mode, gated to exclude microsphere multimers. The instrument calculates the robust mean and the median fluorescence intensity from 100 microspheres of each set. A calibration curve is then constructed for T₄ as described in Bellisario et al., Clin. Chem. 46(9):1422-1424 (2000). A calibration curve for Hb is calculated using LIQUICHIP analysis software (Qiagen Inc., Valencia, Calif., USA). All T₄ and Hb concentrations on the calibration curves are expressed in units of whole blood.

In another approach for measuring Hb, purified human Hb is biotinylated and used in a competitive assay format. For example, a 75 ul aliquot of the dried blood spot eluent diluted 100-fold is applied to each well of a filter assay plate (Millipore MABVN1250). 50 ul of human Hb labeled with biotin is applied to each well. Luminex microspheres conjugated with a pan-Hb antibody (25 ul) are applied and incubated for 60 minutes with shaking at 37° C. The beads are washed three times in phosphate buffered saline/0.05% Tween 20. Streptavidin PE (100 ul, 4 ug/mL) is applied and incubated for 30 minutes with shaking. Beads are washed one time in phosphate buffered saline/Tween 20, resuspended in Luminex sheath fluid and analyzed on the Luminex system.

Dried blood-spot specimens from newborns are then tested following the above protocol. For each specimen, both the hct and T₄ are measured using eluate from the same punch taken from the dried blood-spot.

Computer algorithms provide the calculated values for the concentration of T₄ in each specimen, corrected for the hct. The algorithms use the data obtained from the Hb calibration samples to estimate the hct value for each newborn dried blood-spot specimen, as described in Example 1, which is then used to provide a more accurate, hct-adjusted, T₄ serum concentration value. Importantly, both the hct and the T₄ biomarker are measured from the same punch taken from the same dried blood spot specimen.

### Example 4

The following is a protocol for performing a multiplexed immunoassay measuring the five biomarkers T4 (thyroxine), TSH (thyrotropin), IRT1 and IRT2 (immunoreactive trypsin isoforms 1 and 2), and 17OHP (17-hydroxyprogesterone) and using the Luminex 100 assay (from Luminex Corporation (Austin, Tex.)). Specific examples of the reagents listed in the protocol are provided in following Example 5.

1. Multiplex Assay Protocol

Elute dried blood sample (DBS) standards overnight @23 degrees C., in a shaker @650 rpm in microtiter plate using 100 μL/well of Elution/Assay Buffer.

Transfer 75 μL of the eluates to pre-wet the wells of the filter plate.
Add 25 μL of a mixture of T4 detector antibody and 17OHP tracer to each well.
Add 25 μL of the Luminex bead mix to each well.
Seal with plate sealer, cover, and shake at 37° for 3 hr.
Aspirate, wash wells 3× with 150 μL Wash Buffer.
Resuspend beads in 100 μL of a mixture of TSH detector and Trypsin 1 and Trypsin 2 detector antibodies. Cover, shake at 37° for 1 hr.
Aspirate, wash wells 3× with 150 μL Wash Buffer.
Resuspend beads in 100 μL of 4 μg/mL streptavidin-phycoerythrin (PJRS30, Prozyme).
Cover, shake at 37° C. for 30 min.
Aspirate, wash wells 3× with 150 μL Wash Buffer.
Resuspend beads in 110 μL Sheath Fluid (Luminex cat# 40-50000).
Analyze in Luminex 100 with sample size=80 μL, reading 100 beads of each set, and gate setting=8000 to 13500.

2. Reagent Stocks

DBS Standards/Calibrators and Controls

CDC Multiplex Calibrators: CDC set #6

Microspheres (Beads)

Bead Mix: Beads were coupled according to protocol of manufacturer

(Luminex protocol.

Assay Buffer containing mix of beads conjugated to (a) Thyroxine(T4)-BSA, (b) mAb (monoclonal antibody) to Thyroid Stimulating Hormone(TSH), (c) mAb to human Trypsin 1, (d) mAb to human Trypsin 2 and (e) polyclonal anti-17OHP:
(a) T4-BSA (Fitzgerald 80-IT50); conjugated to Luminex bead 137
(b) mAb to TSH (clone 204-12410) (Meridian (OEM) MAT04-410); conjugated to Luminex bead 136
(c) mAb to human Trypsin 1 (clone 2C4) (Thermo Pierce Ab Shop HYB 021-08-02); conjugated to Luminex bead 183
(d) mAb to human Trypsin 2 (Medix B607); conjugated to Luminex bead 177
(e) polyclonal Ab to 17OHP (Ab 305, from private source); conjugated to Luminex bead 121.

Detector/Reporter Antibodies and Tracer

T4 reporter: Biotinylated mAb to human Thyroxine (OEM Concepts MAT02-525); 0.35 μg/mL)
TSH reporter: Biotinylated mAb to Thyroid Stimulating Hormone (clone M94205, Fitzgerald 10-T25B); 4 μg/mL
Trypsin 1+Trypsin 2 detector mix: Biotinylated rabbit anti-human Trypsin (Biodesign K50900R), 5 μg/ml+Biotinylated anti human Trypsin 1/2/3 (R&D
BAF3586), 1.25 μg/ml
17OHP tracer: Biotinylated 17OHP; use at 1:64,000 dilution
SAPE: Streptavidin-phycoerythrin (Prozyme PJRS30); 4 μg/mL
   T4 reporter, TSH reporter, 17OHP tracer and rabbit anti-human trypsin detector are biotinylated using Thermo Scientific Prod. #21327 No-Weigh Sulfo-NHS-LC-Biotin. Biotinylation is performed according to manufacturer's protocol.

Buffers

Elution/Assay Buffer: The assay buffer contains 9 g of NaCl, 0.5 g of NaN₃, 10 g of BSA, 0.5 g of bovine globulin, 5 g of PEG6000, 0.1 ml Tween 40, 0.5 mg of Danazol, 1 mg of Aprotinin per liter of 50 mM Tris-HCl buffer, pH 7.75.

Wash Buffer: Tris-HCl buffered (7.8) salt solution with Tween 20

### Example 5

A multiplexed immunoassay was performed to measure the five biomarkers T4 (thyroxine) and TSH (thyrotropin), IRT1 and IRT2 (immunoreactive trypsin isoforms 1 and 2), and 17OHP (17-hydroxyprogesterone) in eluates of dried blood samples, to assay for the conditions of congenital hypothyroidism (CH), cystic fibrosis (CF), congenital adrenal hyperplasia (CAH). The assay was performed using the Luminex 100 assay (from Luminex Corporation (Austin, Tex.)). The dried blood samples were obtained from the New York State Department of Health Newborn Screening Laboratory under Institutional Review board Protocol number 07-016. No identifying information was transferred with the specimens.

Multiplex Assay Procedure:

Multiplex standards for each biomarker were obtained from the Centers for Disease Control (CDC). A 3 mm punch from each standard was eluted overnight at 23 C, in shaker at 650 rpm in a microtiter plate using 100 μL/well of elution/assay buffer (containing 9 g of NaCl, 0.5 g of NaN₃, 10 g of BSA, 0.5 g of bovine globulin, 5 g of PEG6000, 0.1 ml Tween 40, 0.5 mg of Danazol, 1 mg of Aprotinin per liter of 50 mM Tris-HCl buffer, pH 7.75). A portion of the eluate (75 μL) was transferred to the wells of a filter plate (which contained a built-in filter used to remove any residual paper fibers dislodged during elution) to pre-wet the wells. The eluate was then filtered prior to use in the assay.

The dried blood spots (a single 3 mm punch per well) were eluted overnight at room temperature in 100 μl of elution buffer with shaking.

To perform the multiplex assay, 75 μl of the sample eluate was combined with 25 μL of T4 detector (biotinylated mAb to human Thyroxine (OEM Concepts MAT02-525); 0.35 μg/mL) and 25 μL of the 17OHP tracer (biotinylated 17OHP; used at a 1:64,000 dilution). The Luminex bead mix (25 μL) was added (the bead mix consisted of the assay buffer containing Luminex beads conjugated to (i) thyroxine(T4)-BSA (Fitzgerald 80-IT50, conjugated to bead 137); (ii) mAb to thyroid stimulating hormone (TSH) (clone 204-12410, Meridian (OEM) MAT04-410, conjugated to bead 136)); (iii) mAb to human Trypsin 1 (human Trypsin 1 (clone 2C4), Thermo Pierce Ab Shop HYB 021-08-02, conjugated to bead 183); and (iv) polyclonal anti-17OHP (polyclonal antibody (Ab) to 17OHP) (Ab 305, private source, conjugated to bead 121). The T4 reporter, TSH reporter, 17OHP tracer and rabbit anti-human trypsin detector were biotinylated using Thermo Scientific Prod. #21327 No-Weigh Sulfo-NHS-LC-Biotin according to the manufacturer's protocol. The microtiter plate was then sealed with plate sealer, covered and shaken at 37 C for 3 hour. Afterwards, each well was aspirated and the beads were washed three times with 150 μL wash buffer (Tris-HCl buffered (pH 7.8) salt solution with Tween 20).

The beads were resuspended in 100 μL of TSH detector (biotinylated mAb to Thyroid Stimulating Hormone (clone M94205 (Fitzgerald 10-T25B), 4 μg/mL), trypsin I and II detector antibody (biotinylated rabbit anti-human trypsin (Biodesign K50900R), 5 ug/ml; and biotinylated anti-human trypsin 1/2/3 (R&D BAF3586), 1.25 ug/ml). The microtiter plate was then covered and shaken for 1 hour at 37 C, and the wells were aspirated and washed three times with 150 μL Wash Buffer.

The beads were then resuspended in 100 μL of 4 μg/mL strep-avidin phycoerythrin (PJRS30, Prozyme). The microtiter plate was then covered and shaken for 30 minutes at 37 C, after which the wells were aspirated and washed three times with 150 μL Wash Buffer. The beads were again resuspended in 110 μL Sheath Fluid (Luminex cat# 40-50000) and analyzed in a Luminex 100 using a sample size of 80 μL, reading 100 beads of each set, with a gate setting of 8000 to 13500.

Before the dried blood sample eluates were assayed for all five biomarkers in the multiplex assay, the assay protocol was validated for each biomarker group/condition:

Validation of Individual Biomarker Assays Using the Multiplex Assay Protocol

### Congenital Hypothyroidism (CH)

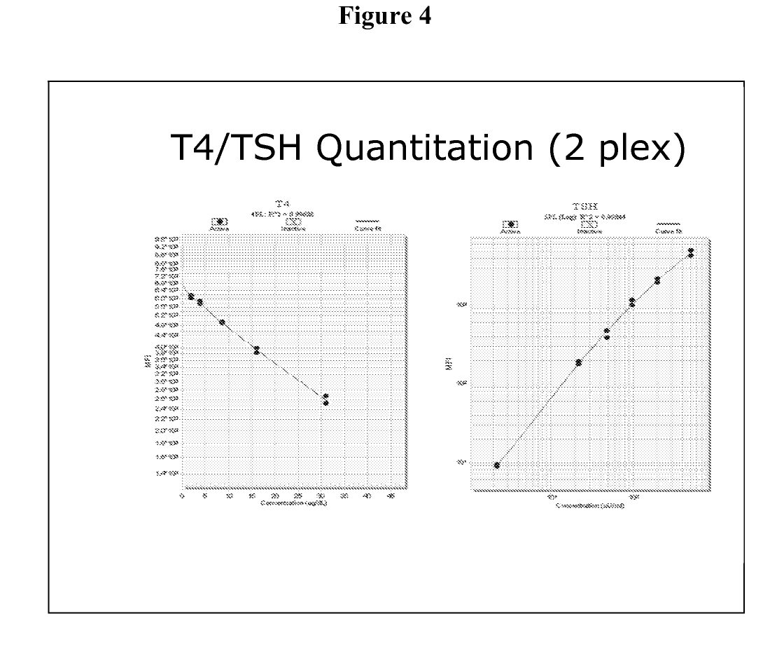

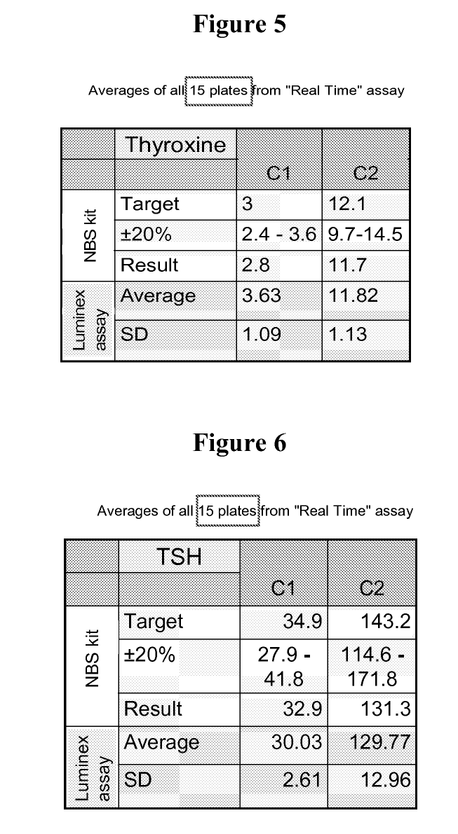

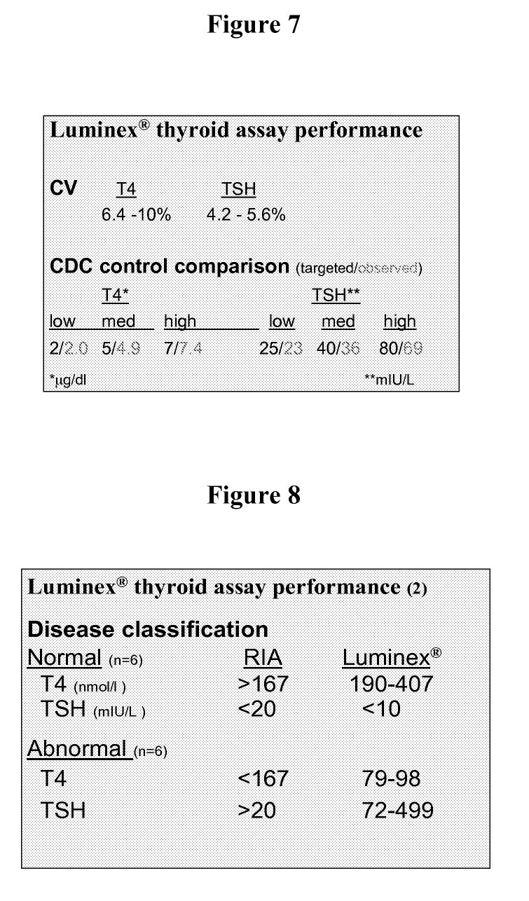

The target biomarkers for CH are thyroxin (T4) and thyrotropin (TSH). FIG. 4 presents the data obtained for the CH validation assay. Included in FIG. 4 are standard curves for each biomarker. The results from assaying control specimens and residual newborn spots are shown in FIGS. 5 and 6. It is noted (see FIGS. 7 and 8) that there is close agreement between the performance of the multiplex assay when compared with these two specimen types.

### Cystic Fibrosis (CF)

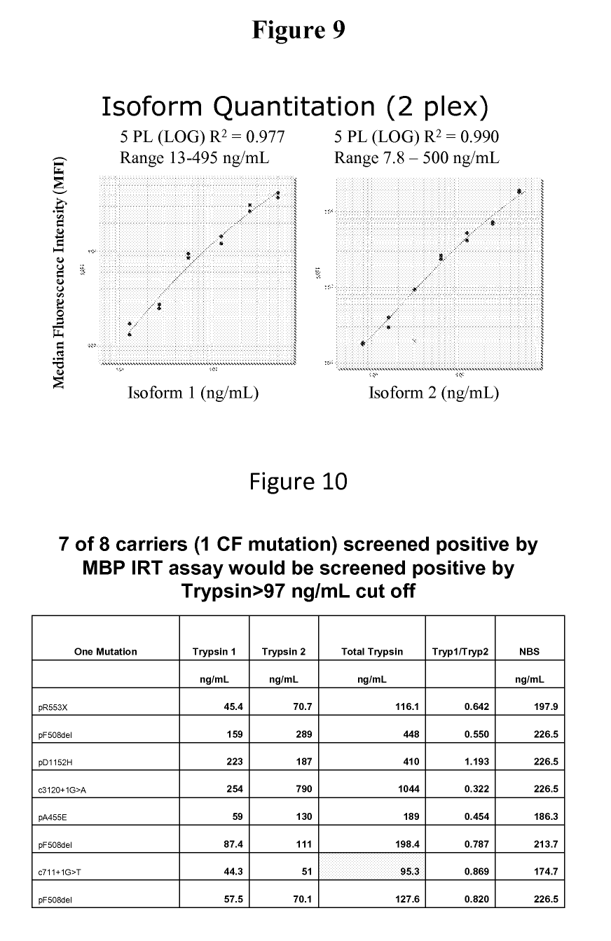

The target biomarkers for CF are two isoforms of immunoreactive trypsin, trypsin 1 and trypsin 2. Data obtained for the CF validation assay are shown in FIGS. 9 and 10. Included in FIG. 9 are standard curves for each biomarker and results from assaying residual newborn spots (FIG. 10) compared with results obtained using the assay currently used in the screening program. In FIG. 10, the performance of the combined IRT1/2 bead sets is compared with results from the newborn screening program. As can be seen from FIG. 10, total trypsin (1+2) identified seven of eight CF carriers. Use of the ratio of these two biomarkers identified seven of eight specimens as carriers. The carrier status in the newborn screening program could only be determined by DNA analysis of the eight specimens. FIG. 10 also demonstrates that there is close agreement between the performance of the multiplex assay when compared with these two specimen types.

### Congenital Adrenal Hyperplasia (CAH)

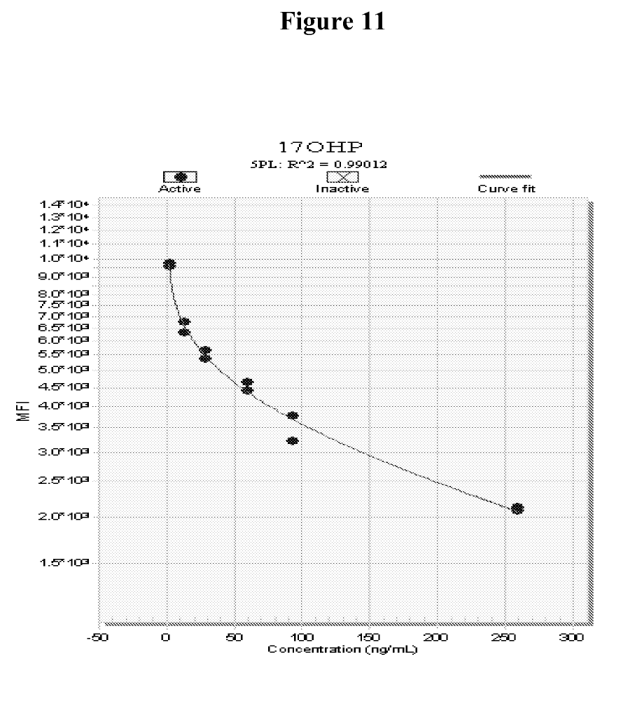

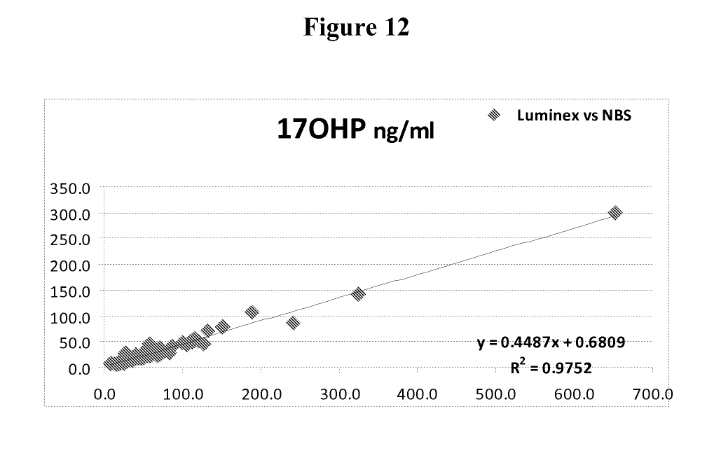

The target biomarker for CAH is 17-hydroxy-progesterone (17OHP), the same as used routinely in newborn screening programs. FIGS. 11 and 12 show the data obtained for the CAH validation assay. Included in FIG. 11 is the standard curve for 17OHP and, in FIG. 12, the results from assaying residual newborn spots compared with the currently used assay in the screening program. In FIG. 12, the values obtained using the Luminex assay (y axis) are plotted as a function of the corresponding values obtained using the currently used assay (x axis). As can be seen from FIG. 12, there is close agreement between the performance of the multiplex assay when compared with values from the screening program.

### Multiplex Assay (CH/CF/CAH)

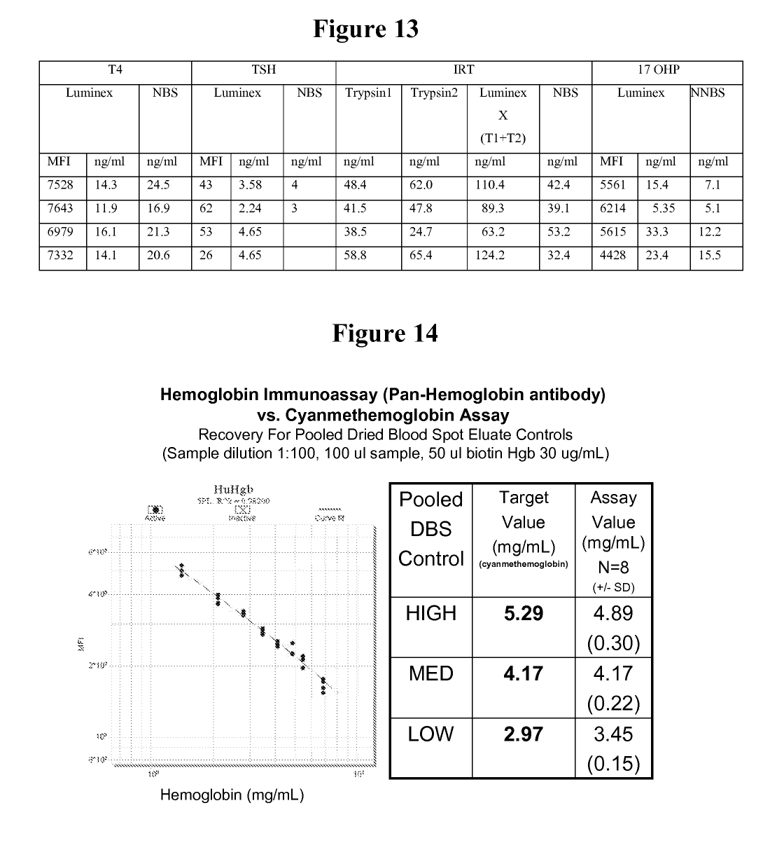

The multiplex assay described above was performed for a series of four residual newborn specimens using a single 3 mm punch taken from each specimen to test for the multiple biomarkers for CH (2 biomarkers), CF(2 biomarkers), and CAH (1 biomarker). FIG. 13 presents a table showing the results of the assay for each specimen, and the corresponding values obtained in the newborn screening program. It can be noted from FIG. 13 that there is close agreement between the performance of the multiplex assay when compared with the values obtained individually in the current newborn screening program.

Determination of Hematocrit

Measurement of total hemoglobin can be used to calculate the hematocrit of a specimen (Kokolm 1991; see FIG. 1). FIG. 14 shows the standard curve used for measurement of total hemoglobin (Hbg's A, F, S, C) in a dried blood specimen and the data derived from that curve used to calculate the hematocrit of the newborn specimen samples used in the multiplex assay. Comparison of these values with the values obtained for the same specimen using the Drabkins reagent show close comparability.

### Example 6

The following describes a series of multiplex assays useful for screening newborns for cystic fibrosis.

Newborn screening for cystic fibrosis (CF) has evolved following the report in 1979 by Crossly et al. (Lancet 1:742-44 (1979)) that blood IRT levels are higher in newborn infants with CF. There are several molecular forms of IRT, the two major forms secreted by exocrine cells of the pancreas are trypsinogen 1 (cationic trypsinogen, IRT1) and trypsinogen 2 (anionic trypsinogen, IRT2) (Guy, O, et al., Biochemistry 17(9):1669-75 (1978); Kimland, M, et al., Clinica Chimica Acta 184:31-46 (1989)). Normally the IRT1 form is present in higher levels, however in pathological conditions such as pancreatitis the IRT2 form becomes predominant (Itkonen, O, et al., J. Lab. Clin. Med. 115:712-8 (1990)). Today 46 states provide NBS for CF, all using IRT for the initial screen. For the year 2007, the most recent year with complete data, 9,076 infants were screen positive and 300 confirmed with CF, a ratio of 30:1 screen positive to confirmed CF. (http://www2.uthscsa.edu/nnsis/). Subsequent testing after an initial screen positive can use a number of different protocols (Wilcken, B., J. Inherit. Metab. Dis. 30:537-543 (2007)) in an effort to minimize the number of false positive results, such as IRT positives tested again on a newly collected specimen; IRT with DNA analysis on that same first specimen, and others.

A number of investigators have developed immunoassays to IRT, and commercial assays currently in use have employed both monoclonal and polyclonal antibodies for IRT (Deam, S M, et al., Wein Klin Wochenschr 100:55-7 (1988); Cabrini, G., et al., Clin. Biochem. 23:213-19 (1990); and Ball, C L, et al., Clin. Chem. Lab. Med. 43(5):570-572 (2005)). The heterogeneous nature of IRT and differing specificity of antibodies to the various components have raised issues with the standardization and external QC of the assay. As noted by Li et al (Li, L, et al., Journal of Medical Screening 13:79-84 (2006)), the lack of a universally acceptable IRT standard has made the comparison of absolute IRT values among commercial immunoassays difficult. As reported by Lafont (Lafont, P, et al., Clinica Chimica Acta 235:197-206 (1995)) trypsinogen exists in many forms in the serum. These different forms of trypsinogen are not recognized equally among immunoassays, thus contribute to the discordant results when comparing assay to assay.

In the present study we report development of a suspension array multiplexed immunoassay for the two specific isoforms of trypsinogen IRT1 and IRT2. The specificity of the assay for the two isoforms allows development of external QC for the heterogeneous forms of IRT and allows for analysis of the IRT1:IRT2 ratio as a potential added parameter before referral for mutation analysis.

### Materials and Methods

### Antibody Reagents

Anti-trypsin isoform specific monoclonal antibodies were coupled to Luminex xMAP microspheres following the manufacturers protocol. IRT1 capture monoclonal antibody HYB 021-08-02 was obtained from Affinity Bioreagents, the IRT2 capture monoclonal 8607 was obtained from Medix Biochemica. Polyclonal detector antibody K50900R (Biodesign International) was biotinylated using the Fluoreporter biotin-XX labeling kit (Invitrogen) according to manufacturers instructions. A sheep polyclonal anti-trypsin 1/2/3 BAF3586 was purchased with the biotin label from the manufacturer (R&D Systems). The two antibodies were combined to make the detector mix, with K50900 at a concentration of 5.0 ug/mL and BAF3586 at 1.25 ug/mL.

### Assay Calibrators

IRT1 calibrators were used from the comparison reference method kit (MPBiomedical). IRT2 calibrators were prepared from recombinant IRT2 (R&D Systems). Serum was treated with activated charcoal according to the method of Li et al (Li, L, et al., Journal of Medical Screening 13:79-84 (2006)) and combined with washed red blood cells to make whole blood at 55% hematocrit. Aprotinin (Sigma Chemical) was added at a concentration of 1 mg/L. The reconstituted whole blood was enriched with the recombinant IRT2 and dispensed to make the dried blood spot calibrators. The spots were air dried overnight, packed with desicant and stored frozen at −20 deg. C.

### Assay Procedure

Assay buffer was prepared containing phosphate buffered saline (Sigma Chemical Co.), 0.055 Tween 20, 0.05% sodium azide, 0.2% gelatin. 1 mg/L aprotinin (Sigma Chemical Co.) was added to the assay buffer to prepare the spot elution buffer. The dried blood spots (a single 3 mm punch per well) were eluted overnight at room temperature in 100 ul of elution buffer with shaking. For the assay, 75 ul of the sample eluate was combined with 25 ul of the trypsin 1 and trypsin 2 bead mix in order to obtain 2000 microspheres per well for each of the analytes. The capture incubation was for 3 hours at 37 deg. C. with shaking Microspheres were washed three times in 100 ul assay buffer, and 100 ul of the anti trypsin detector antibody mix was added to each well. The detector antibodies were incubated for 1 hour at 37 deg. C. with shaking, and the microspheres were again washed three times with 100 ul assay buffer. For detection, 100 ul of streptavidin PE (Invitrogen, 5-866) was added at 4 ug/mL, and incubated for 30 minutes at 37 deg. C. The assay plate was aspirated and the microspheres resuspended in 100 ul of Luminex sheath fluid for analysis.

### Samples

All clinical samples assayed were obtained from the New York State Dept. of Health Newborn Screening Laboratory under an Institutional Review Board Protocol number 07-016. No identifying information was transferred with the specimens.

### Results

### Correlation

The selection criteria for the samples analyzed in the correlation study are shown in TABLE 1:

TABLE 1
Correlation Study Sample
Selection Criteria

| Reference IRT | Sample |
|---|---|
| value range ng/mL | N |
| IRT < 35 | 32 |
| IRT 35-55 | 32 |
| IRT 55-100 | 32 |
| IRT 100-170 | 40 |
| IRT > 170 | 32 |

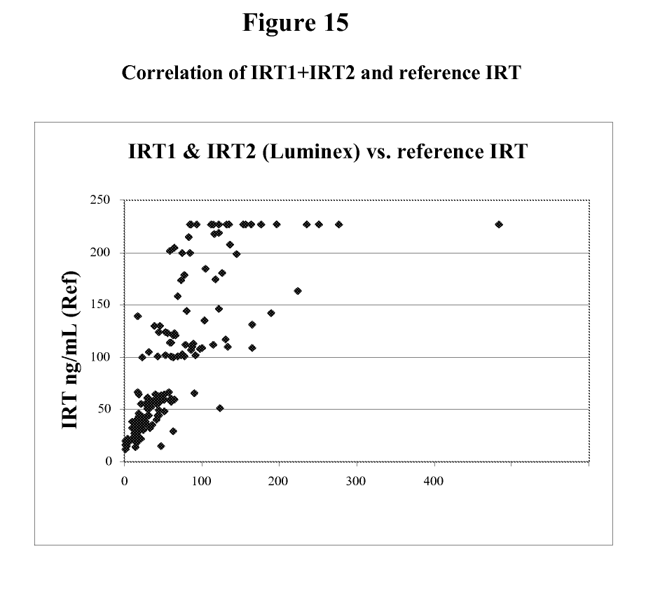

IRT1+IRT2 (IRT1−IRT2) screen negative by the IRT1 and IRT2 assay were compared with the IRT-R and had a correlation coefficient of 0.75 (see FIG. 15), with a mean value lower than the IRT1, IRT1/IRT2: X=63.8+/−SD 62.6, the IRTR: X=92.9+/−SD 69.0. This is not surprising due to the extremely different formats of the assays and different antibodies. (11) Of the 133 samples that were screen negative by the IRTR assay, 11 were screen positive using the IRT1/IRT2 assay. Having no link back to the specimen, it was impossible to retest and verify these findings. Of the 32 cases screen positive by the IRTR, 11 cases were screen negative by the IRT1 and IRT2 method. We have no way to show a screen negative or positive by IRT2 alone. Each of these screen negative cases had been confirmed to have no CF mutations by the screening program in its second tier mutation analysis.

### Screen Positive Sample Evaluation

The screen positive sample population consisted of ten confirmed positive cases with 2 CF mutations; six cases with two CF mutations, disease not yet confirmed; eight cases with 1 CF mutation; and, 137 cases with no CF mutations detected. The screen positive cut-off established by the New York DOH NBS laboratory for the reference method is a value greater than 170 ng/mL including the top 5% of each assay. The screen positive sample evaluation shown in TABLE 2 indicated that a total trypsin (sum of IRT1 and IRT2) cut off >97 ng/mL would be necessary to achieve 100% sensitivity for the confirmed disease population. Calculation of the IRT1:IRT2 ratio for this population indicated that a ratio <2.0 is also consistent with the elevated IRTR value.

TABLE 2
Screen Positive Sample Evaluation

| | | | | | Refer- |
|---|---|---|---|---|---|
| | Trypsin | Trypsin | Total | Tryp1/ | ence |
| Two Mutations | 1 | 2 | Trypsin | Tryp2 | IRT |
| Confirmed Disease | Ng/mL | Ng/mL | ng/mL | ng/mL | ng/mL |
| Del F508/3121 + G > A | 121 | 129 | 250 | 0.938 | 248 |
| Del F508/Del F508 | 63.6 | 93.2 | 156.8 | 0.682 | 183.5 |
| Del F508/Del F508 | 117 | 184 | 301 | 0.636 | 248 |
| Del F508/R553X | 104 | 269 | 373 | 0.387 | 248 |
| Del F508/Del F508 | 96.6 | 103 | 199.6 | 0.938 | 248 |
| Del F508/W1282X | 265 | 326 | 591 | 0.813 | 248 |
| Del F508/Del F508 | 131 | 129 | 260 | 1.016 | 248 |
| Del F508/N1303K | 111 | 130 | 241 | 0.854 | 248 |
| Del F508/Undetected | 154 | 354 | 508 | 0.435 | 248 |
| Del F508/R117H, 7T, | 67.6 | 33.6 | 101.2 | 2.012 | 194.3 |
| 9T, var | | | | | |
| pF508/pF508 | 95.4 | 119 | 214.4 | 0.802 | 248 |
| Del pE60x/Del F508 | 40.1 | 57.1 | 97.2 | 0.702 | 248 |
| p.S549/c387delA | 76.8 | 107 | 183.8 | 0.718 | 248 |
| pR117H/pD1152H, | 107 | 364 | 471 | 0.294 | 226.5 |
| 7T, 7T | | | | | |
| Del pF508/Del pF508 | 78.4 | 62.4 | 140.8 | 1.256 | 226.5 |
| pG85E/pF508del | 252 | 884 | 1136 | 0.285 | 226.5 |

TABLE 3 shows the analysis of eight single mutation CF carriers that were screen positive by the IRTR assay. Seven of these carriers would also screen positive by use of the IRT1/IRT2 cut off of 97 ng/mL. In the 137 cases that were screen positive by the IRTR assay with no CF mutations detected, 26 would be screen negative using the IRT1/IRT2 cut off of >97 ng/mL, a reduction of 19% in the false positive rate in this selected study population.

TABLE 3
Carriers (1 CF mutation) Screen Positive by IRTR

| | Trypsin | Trypsin | Total | | |
|---|---|---|---|---|---|
| | 1 | 2 | Trypsin | Tryp1/ | |
| One Mutation | ng/mL | ng/mL | ng/mL | Tryp2 | reference |
| pR553X | 45.4 | 70.7 | 116.1 | 0.642 | 197.9 |
| pF508del | 159 | 289 | 448 | 0.550 | 226.5 |
| pD1152H | 223 | 187 | 410 | 1.193 | 226.5 |
| c3120 + 1G > A | 254 | 790 | 1044 | 0.322 | 226.5 |
| pA455E | 59 | 130 | 189 | 0.454 | 186.3 |
| pF508del | 87.4 | 111 | 198.4 | 0.787 | 213.7 |
| c711 + 1G > T | 44.3 | 51 | 95.3 | 0.869 | 174.7 |
| pF508del | 57.5 | 70.1 | 127.6 | 0.820 | 226.5 |

*cut up >97 ng/ml
**cutoff >2.0

Screen negative samples with confirmed disease Analysis of three cases of confirmed disease with an IRTR value below the 170 ng/mL cut off is shown in TABLE 4. Two of the three cases would be screen positive using the IRT1/IRT2 assay criteria of total IRT.

TABLE 4
Confirmed disease with reference IRT <170 ng/mL

| Confirmed | Trypsin | Trypsin | Total | Tryp1/ | Ref |
|---|---|---|---|---|---|
| Disease | 1 | 2 | Trypsin | Tryp2 | IRT |
| 2 mutations | ng/mL | ng/mL | ng/mL | ng/mL | ng/mL |
| W1282X/N1303K | 55.2 | 49.8 | 105 | 1.108 | 147.6 |
| Del508/Del508 | 28.1 | 48.7 | 76.8 | 0.577 | 67.9 |
| Del508/Del508 | 38.5 | 73.8 | 112.3 | 0.522 | 111.8 |

### Population Study

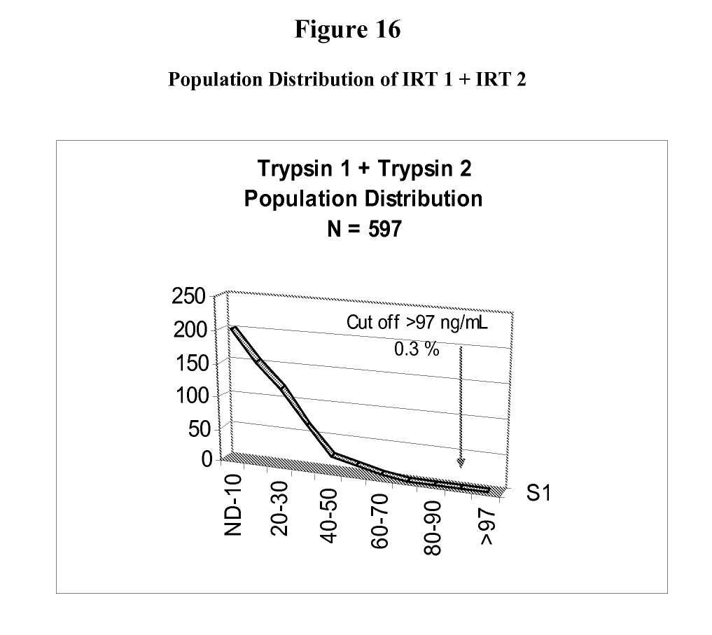

A total of 597 population study samples were analyzed, the distribution is shown in FIG. 16. Two cases in this population were screen positive by the IRT1-IRT2 criteria, of these one case fell within the top 5% of the reference IRT method and had 1 CF mutation detected, the second case was screen negative by the reference IRT.

The screen false positive rate in newborn screening for CF has remained persistently high, despite numerous attempts to lower it. (Wilcken, B., J. Inherit. Metab. Dis. 30:537-543 (2007)) One unexplored approach, use of the two isoforms of trypsin, was examined in these studies. The goal in this study was the development of a multiplexed assay for CF using the two major trypsinogen isoforms that would meet screening standards for clinical accuracy in comparison to current commercial IRT assays.

The correlation study showed substantially equivalent performance of the assays in segregation of a screen positive population. Importantly for the 11 discrepant cases that were screen positive in the IRTR but screen negative in the IRT1/IRT2 assay, no CF mutations were detected in them by the screening program as part of its protocol, suggesting a greater sensitivity for the multiplex assay. Analysis of a screen positive population with confirmed disease indicated that a cut off of >97 ng/mL in the IRT1/IRT2 assay would be needed to achieve 100% sensitivity for these samples. Although this cut off is substantially lower than the one developed for the reference method of 170 ng/mL, it is nearer to one of 112 ng/mL reported for a monoclonal antibody based method for total IRT. (Ball, C L, et al., Clin. Chem. Lab. Med. 43(5):570-572 (2005)). Li, et al. (Li, L, et al., Journal of Medical Screening 13:79-84 (2006)) reported that the measured immunoreactivity of an IRT spiked preparation was 45%-60% of that specified by the company providing the material. It is possible that more specific immunoreactivity is observed when measuring the two isoforms separately as reported here.

Cystic fibrosis carriers have been shown to have higher IRT values than the normal population (Casellani, C, et al., Am. J. Med. Genet. A 135(2):142-4 (2005); Lecoq, I, et al., Acta Paediatr. 88:338-341 (1999)). In a screening program in which the goal is detect disease and not carrier status, correct identification of carrier status could be a great help. In these studies use of the total IRT1-IRT2 was unable to discriminate the carrier population, with seven of eight carriers screen positive by the reference assay also screen positive by the IRT1/IRT2 criteria.

In three cases identified with confirmed disease that had a reference IRT below the cut off, two were screen positive by the IRT1/IRT2 criteria. More studies are needed to determine whether these results indicate that the IRT1/IRT2 assay has greater specificity.

This study demonstrates that the IRT1/IRT2 multiplexed assay for CF has substantial equivalence in detecting screen positive specimens compared with the reference IRT method. The specificity of the antibodies for the two isoforms would also provide advantages in the standardization and external QC of the assay. Perhaps more importantly, the multiplex format will allow additional biomarker e.g., PAP (Sarles, J, et al., J. Pediatr. 147:302-5 (2005)), to be added in the future. An even more optimistic goal is the combining of this CF assay with immunoassays for congenital hypothyroidism, and congenital adrenal hyperplasia into a single assay for the three, thereby saving time in a screening laboratory specimen usage, and perhaps at a lower cost.

All publications cited in this specification are herein incorporated by reference in their entirety to the same extent as if each individual document was specifically and individually indicated to be incorporated by reference in its entirety. While the invention has been described with reference to a particularly preferred embodiment, it will be appreciated that modifications can be made without departing from the spirit of the invention. Such modifications are intended to fall within the scope of the appended claims.

1. A method for determining a concentration of a biomarker in the serum of a patient, said method comprising:
(a) obtaining a blood sample from a patient;
(b) obtaining an extract from the blood sample;
(c) measuring a concentration of hemoglobin (Hb) in the extract;
(d) calculating a hematocrit (hct) value for the blood sample from the measured concentration of Hb;
(e) measuring a concentration of a biomarker in the extract; and
(f) determining a concentration of the biomarker in the serum of the patient using the calculated het value, volume of the blood sample, and the measured concentration of biomarker in the extract.

2. The method of claim 1, wherein the blood sample is a dried blood sample.

3. The method of claim 1, wherein a computer algorithm is used to determine the concentration of the biomarker in the serum of the patient using the calculated hct value, volume of the blood sample, and the measured concentration of biomarker in the extract.

4. The method of claim 1, wherein the biomarker is a metabolic biomarker.

5. The method of claim 4, wherein the metabolic biomarker is selected from the group consisting of T4, TSH, and an amino acid.

6. The method of claim 1, further comprising (g) measuring a concentration of a second biomarker in the extract and determining a concentration of the second biomarker in the scrum of the patient using the calculated hct value, volume of the blood sample, and the measured concentration of the second biomarker in the extract.

7. The method of claim 4, wherein the metabolic biomarker is suitable for use in a newborn screening program.

8. The method of claim 2, wherein the patient is a human infant.

9. The method of claim 1, wherein step (c) is conducted using an affinity-based assay.

10. The method of claim 9, wherein the affinity-based assay comprises an immunoassay.

11. The method of claim 1, wherein step (e) is conducted using an affinity-based assay.

12. The method of claim 11, wherein the affinity-based assay comprises an immunoassay.

13. The method of claim 1, wherein steps (c) and (e) are conducted using a multiplexed immunoassay.

14. The method of claim 1, further comprising identifying whether said patient possesses an aberrant hct level.

15. The method of claim 14, wherein said aberrant hct level is indicative of anemia.

16. A method of calculating a concentration of a biomarker in the serum of a patient, the method comprising:
(a) measuring a concentration of Hb and a concentration of a biomarker in an extract obtained from a dried blood sample from a patient;
(b) converting the measured concentration of Hb to a hct value; and
(c) calculating a concentration of the biomarker in the serum of the patient using the hct value and the measured concentration of the biomarker in the extract.

17. A method of arriving at concentrations of one or more biomarkers in a patient's whole blood, the method comprising:
(a) obtaining a punch from a dried whole blood specimen of a patient, wherein the dried whole blood specimen is made from a whole blood sample taken from the patient;
(b) obtaining an eluent from the punch;
(c) measuring concentrations of one or more biomarkers and of Hb in the eluent or a diluent thereof;
(d) estimating an het fraction of the whole blood sample from the measured concentration of Hb; and
(e) adjusting the measured concentrations of the one or more biomarkers based on the estimated hct fraction to arrive at concentrations of the one or more biomarkers in the serum fraction of the patient's whole blood.

18. The method of claim 17, wherein the measuring step is conducted using a multiplexed assay system, which measures substantially simultaneously the concentrations of at least the one or more biomarkers.

19. A method of arriving at concentrations of one or more biomarkers in a patient's whole blood using measurements obtained from a dried whole blood specimen, the method comprising:
(a) measuring concentrations of one or more biomarkers in an eluent from a punch of a dried whole blood specimen made from a whole blood sample taken from a patient;
(b) measuring, a concentration of Hb in the eluent or a diluent thereof;
(c) estimating an hct fraction for the whole blood sample from the measured concentration of Hb; and
(d) adjusting the measured concentrations of the one or more biomarkers based on the estimated hct fraction to arrive at concentrations of the one or more biomarkers in the patient's whole blood.

20. A method for determining a concentration of Hb in a sample, said method comprising:
(a) contacting the sample with a fluorogenic substrate for peroxidase and hydrogen peroxide, such that when Hb is present a fluorescent product is produced;
(b) determining the amount of the fluorescent product; and
(e) calculating the concentration of the Hb in the sample based upon the amount of the fluorescent product determined in (b).

21. The method of claim 20, wherein the sample is an extract of a blood sample obtained from a patient.

22. The method of claim 20, wherein the substrate is AMPLEXRED or AMPLEX ULTRARED.

23. The method of claim 21, further comprising
(a) calculating a hct value for the blood sample using the concentration of the Hb in the extract;
(b) measuring a concentration of a biomarker in the extract; and
(c) determining a concentration of the biomarker in plasma of the blood sample from the patient using the calculated hct value, volume of the blood sample, and the concentration of the biomarker in the extract.

24-27. (canceled)

28. An assay buffer for the substantially simultaneous analysis of two or more biomarkers, the buffer comprising, in an aqueous mixture: (i) tris(hydroxymethyl)aminomethane hydrochloride (Tris-HCl); (ii) sodium chloride (NaCl); (iii) one or more emulsifiers; (iv) bovine serum albumin (BSA); (v) polyethylene glycol (PEG); (vi) one or more preservatives; (vii) bovine globulin; and (viii) a testosterone derivative.

29-42. (canceled)

43. An aqueous buffer for the substantially simultaneous analysis of two or more biomarkers, the buffer comprising, in an aqueous mixture: (i) about 50 mM Tris-HCl; (ii) about 150 mM NaCl; (iii) about 0.02% Tween 40; (iv) about 1% BSA; (v) about 0.5% polyethylene glycol; (vi) a preservative; (vii) about 0.05% bovine globulin; (viii) about 0.5 mg/L danazol; and (ix) about 1 mg/L of a protease inhibitor; provided that the assay buffer does not include about 0.5 mg or more per liter of 8-anilino-1-naphthalenesulfonic acid or a salt thereof.

44-51. (canceled)

52. A method of arriving at concentrations of two or more biomarkers in a serum portion of a whole blood sample taken from a patient, the method comprising:
(a) obtaining one or more punches from a dried whole blood specimen of a patient, in which the dried whole blood specimen is made from a whole blood sample taken from the patient;
(b) obtaining an eluent from the one or more punches using a universal buffer as an elution solvent;
(c) measuring a concentration of each of two or more biomarkers and of hemoglobin (Hb) in the eluent or a diluent thereof, provided that the measurement of the concentration of each of the two or more biomarkers is carried out substantially simultaneously using a multiplexed affinity assay;
(d) estimating a hematocrit (het) value for the whole blood sample from the concentration of Hb measured in step (c);
(e) using the estimated het value to adjust the concentration of each of the two or more biomarkers measured in step (c) to arrive at concentrations of two or more biomarkers in a serum portion of the whole blood sample taken from the patient.

53-71. (canceled)

72. A method of determining whether a patient has, or may develop, two or more disorders by using a multiplexed affinity assay to determine the concentrations of two or more biomarkers indicative of the disorders in a serum portion of a whole blood sample taken from the patient, the method comprising:
(a) obtaining a punch from a dried whole blood specimen of a patient, in which the dried whole blood specimen is made from a whole blood sample taken from the patient;
(b) obtaining an eluent from the punch using a universal buffer as an elution solvent;
(c) measuring a concentration of each of two or more biomarkers and of total hemoglobin (Hb) in the eluent or a diluent thereof, provided that the measurement of the concentration of each of the two or more biomarkers is carried out substantially simultaneously using a multiplexed affinity assay;
(d) estimating a hematocrit (hct) value for the whole blood sample from the concentration of total Hb measured in step (c);
(e) using the estimated het value to adjust the concentration of each of the two or more biomarkers measured in step (c) to arrive at adjusted concentrations of the two or more biomarkers in a serum portion of the whole blood sample taken from the patient; and
(f) using the adjusted concentrations of the two or more biomarkers to determine whether the patient has, or may develop, two or more disorders.

73-78. (canceled)
